SEQUENCE LISTING (15 sequences)

SEQ ID NO: 1 - protein, 343 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 2 - DNA, 22 bp, synthetic construct
  source 1..22: mol_type = other DNA
aggagctgtcattagggacatc

SEQ ID NO: 3 - DNA, 22 bp, synthetic construct
  source 1..22: mol_type = other DNA
ctccaagccaaagtccttagag

SEQ ID NO: 4 - DNA, 20 bp, synthetic construct
  source 1..20: mol_type = other DNA
aagcagctgaatgggaggac

SEQ ID NO: 5 - DNA, 20 bp, synthetic construct
  source 1..20: mol_type = other DNA
tagatgcgatggtgtcgagc

SEQ ID NO: 6 - DNA, 20 bp, synthetic construct
  source 1..20: mol_type = other DNA
ctacccaggggctgcttatc

SEQ ID NO: 7 - DNA, 20 bp, synthetic construct
  source 1..20: mol_type = other DNA
agcgggggttaaagtggaag

SEQ ID NO: 8 - DNA, 20 bp, synthetic construct
  source 1..20: mol_type = other DNA
tggaggcattctcatgccag

SEQ ID NO: 9 - DNA, 20 bp, synthetic construct
  source 1..20: mol_type = other DNA
aagacacagtgtggtcctcg

SEQ ID NO: 10 - DNA, 20 bp, synthetic construct
  source 1..20: mol_type = other DNA
gggctcttgggaactacacg

SEQ ID NO: 11 - DNA, 20 bp, synthetic construct
  source 1..20: mol_type = other DNA
agactgtactcgggctctga

SEQ ID NO: 12 - DNA, 20 bp, synthetic construct
  source 1..20: mol_type = other DNA
cttgctggaaccgtcaccat

SEQ ID NO: 13 - DNA, 20 bp, synthetic construct
  source 1..20: mol_type = other DNA
ctcttgattcctggaggtgc

SEQ ID NO: 14 - DNA, 20 bp, synthetic construct
  source 1..20: mol_type = other DNA
cgcggttctattttgttggt

SEQ ID NO: 15 - DNA, 20 bp, synthetic construct
  source 1..20: mol_type = other DNA
agtcggcatcgtttatggtc

(19) United States
(12) Patent Application Publication
(10) Pub. No.: US 2024/0264149 A1
(43) Pub. Date: Aug. 8, 2024

(54) COMPOSITION FOR PREVENTING OR TREATING OF ISCHEMIC CEREBROVASCULAR DISEASE COMPRISING AN ARGINASE-1 INHIBITOR

(71) Applicant: Ajou University Industry-Academic Cooperation Foundation, Gyeonggi-do (KR)

(72) Inventors: Byung Gon KIM, Seoul (KR); Hyung Soon KIM, Gyeonggi-do (KR)

(73) Assignee: Ajou University Industry-Academic Cooperation Foundation, Gyeonggi-do (KR)

(21) Appl. No.: 18/472,987

(22) Filed: Sep. 22, 2023

(30) Foreign Application Priority Data
Feb. 2, 2023 (KR) 10-2023-0014510

(51) Int. Cl. G01N 33/50; A61K 45/06; A61P 25/28; C12N 15/113; G01N 33/68

(52) U.S. Cl. CPC G01N 33/5023; A61K 45/06; A61P 25/28; C12N 15/1137; G01N 33/5058; G01N 33/6863

(57) ABSTRACT

The present disclosure relates to a composition for preventing or treating ischemic cerebrovascular disease, and more specifically to a composition for preventing or treating ischemic cerebrovascular disease including an arginase-1 inhibitor as an active ingredient, and the arginase-1 inhibitor according to the present disclosure can alleviate pathological damage caused by the interaction of inflammatory cells after ischemic stroke and promote functional recovery by inhibiting the expression and function of cell-specific arginase-1 protein.

### BACKGROUND

### 1. Field of the Invention

The present disclosure relates to a composition for preventing or treating ischemic cerebrovascular disease, and more specifically to a composition for preventing or treating ischemic cerebrovascular disease including an arginase-1 inhibitor as an active ingredient.

### 2. Discussion of Related Art

In vertebrates, the central nervous system, which is divided into the skull and spinal cord, has very limited recovery after injury. Normally, brain tissue receives a large amount of blood flow, but if blood flow is impaired due to a blood clot, it can cause severe damage to the central nervous system such as ischemic cerebrovascular disease. Ischemic cerebrovascular disease causes acute cerebral infarction in which nervous tissue is damaged, and cerebral infarction causes headache, dizziness, visual impairment, speech impairment, motor impairment and hemiparesis. In addition, it is difficult to expect a good prognosis after the onset of cerebral infarction due to the limited regenerative capacity of the central nervous system and limited treatment methods.

To date, treatments for ischemic cerebrovascular disease have relied on the administration of tPA (tissue plasminogen activator) or thrombectomy. In particular, tPA, which is used as a therapeutic agent for ischemic cerebrovascular disease, has a very limited therapeutic window, and thus, it can only be applied to patients with acute stroke.

Recent studies on the treatment of ischemic cerebrovascular disease show that the inflammatory response induced after cerebral infarction plays a major role in functional recovery after stroke. In particular, the creation of an inflammatory environment through the interaction between infiltrating macrophages and microglia inflammatory cells that are involved in the inflammatory response is attracting great attention.

### SUMMARY OF THE INVENTION

An object of the present disclosure is to reveal the function of arginase-1 protein that is expressed in inflammatory cells after ischemic cerebrovascular disease through a study on inhibition of the function thereof, and to provide a mechanism that can be a treatment target after damage from ischemic cerebrovascular disease.

In addition, another object of the present disclosure is to apply an orally administrable arginase-1 protein inhibitor on the same principle as cell-specific arginase-1 protein inhibition to provide a method of utilizing an arginase-1 inhibitor for the treatment of ischemic cerebrovascular disease.

The present disclosure provides a pharmaceutical composition for preventing or treating ischemic cerebrovascular disease, including an arginase-1 inhibitor as an active ingredient.

In addition, the present disclosure provides a method for screening a substance for preventing or treating ischemic cerebrovascular disease, including the steps of (a) treating stroke-inducing mice with a test substance to induce the deletion of a gene encoding the arginase-1 protein or the inhibition of arginase-1 protein function: (b) measuring the inflammatory cytokine expression rate in stroke-induced mice in which a gene in the arginase-1 protein is defective or the function of the arginase-1 protein is inhibited; and (c) selecting a candidate substance that reduces the expression rate of inflammatory cytokines in stroke-induced mice that are treated with the test substance compared to stroke-induced mice that are not treated with the test substance.

The arginase-1 inhibitor according to the present disclosure can alleviate pathological damage caused by the interaction of inflammatory cells after cerebral infarction and promote functional recovery by suppressing the expression or inhibiting the function of the cell-specific arginase-1 protein.

Therefore, the composition including the same as an active ingredient can be advantageously used as a therapeutic agent for the prevention or treatment of ischemic cerebrovascular diseases such as cerebral infarction through arginase-1 inhibition, and it can also be advantageously used as a therapeutic agent for ischemic cerebrovascular disease by administering an oral arginase-1 inhibitor that acts by the same mechanism.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1A is a mimetic diagram of the construction of a cerebral infarction model to confirm the expression pattern of arginase-1 protein in the photothrombotic cerebral infarction model, and FIG. 1B shows the expression of arginase-1 protein over time through tissue immunostaining;

FIGS. 2A and 2B show the results of confirming cells expressing arginase-1 protein through tissue immunostaining in a cerebral infarction model;

FIG. 3A is a mimetic diagram of the cre-loxp system for specifically suppressing the expression of arginase-1 protein in infiltrating macrophages, FIGS. 3B and 3C show the results of confirming that the expression of arginase-1 is suppressed in the corresponding animals, FIGS. 3D and 3E show the results of tissue immunostaining, FIGS. 3F and 3H show the results of the PCR experiment, and FIG. 3G shows the results of the Western blot experiment;

FIGS. 4A and 4B show the results of a behavioral test (mNSS scoring) to evaluate functional recovery after cerebral infarction in animals lacking the arginase-1 gene;

FIGS. 5A and 5B show the results of a behavioral experiment (pellet experiment) to evaluate functional recovery after cerebral infarction in animals lacking the arginase-1 gene;

FIGS. 6A and 6B show the results of a behavioral experiment (cylinder experiment) to evaluate functional recovery after cerebral infarction in animals lacking the arginase-1 gene;

FIGS. 7A and 7B show the results of a behavioral experiment (ladder test) to evaluate functional recovery after cerebral infarction in animals lacking the arginase-1 gene;

FIG. 8A is a mimetic diagram showing changes in the level of inflammatory signal molecules in isolated microglia, and FIGS. 8B to 8D show genetic phenotypic changes in microglia around the lesion after cerebral infarction in arginase-1 gene deficient animals;

FIGS. 9A to 9F show pathological changes in the cerebral tissue of arginase-1 gene deficient animals after cerebral infarction; and

FIGS. 10A to 10C confirm synapse formation around the lesion after cerebral infarction in arginase-1 gene-deficient animals.

### DETAILED DESCRIPTION OF EXEMPLARY EMBODIMENTS

Hereinafter, the present disclosure will be described in detail.

The inventors of the present disclosure attempted to discover a treatment target for ischemic cerebrovascular disease by inhibiting a protein that is specifically expressed in macrophages infiltrating tissues after cerebral infarction, and in this study, the present disclosure was completed by confirming that the inhibition of the arginase-1 protein originating from infiltrating macrophages can dramatically increase the recovery of synaptic structure and motor function after cerebral infarction.

Therefore, in the present disclosure, in order to specifically suppress the expression of arginase-1 protein in infiltrating macrophages in an animal model of cerebral infarction, after the arginase-1 gene was induced to be specifically depleted in infiltrating macrophages expressing LysM, functional recovery after cerebral infarction was evaluated. The composition including the arginase-1 inhibitor of the present disclosure can provide a basis for use as a useful therapeutic agent for patients with ischemic cerebrovascular disease.

Therefore, the present disclosure provides a model for the cell-specific inhibition of arginase-1 protein expression in photothrombotic cerebral infarction model mice, and presents the concept of using an oral arginase-1 inhibitor as a therapeutic agent for ischemic cerebrovascular disease.

The present disclosure relates to a pharmaceutical composition for preventing or treating ischemic cerebrovascular disease, including an arginase-1 inhibitor as an active ingredient.

Arginase-1 in the present disclosure refers to a protein that regulates the inflammatory response of infiltrating macrophages after cerebral infarction.

The expression of arginase-1 in the present disclosure may be suppressed by deleting a gene encoding the arginase-1 protein or inhibiting the function of the arginase-1 protein.

The gene deletion means that one or more bases in the gene are removed, which means that the activity of the protein encoded by the gene is inactivated or the protein encoded by the gene is not synthesized.

Inhibiting the function of arginase-1 protein may mean inhibiting the function at the protein level and inhibiting the catalytic activity of the arginase-1 enzyme protein.

The arginase-1 inhibitor in the present disclosure is any substance that can cause defects in the expression of arginase-1 gene or inhibit the function of arginase-1 protein, and it is not limited to the type or kind of substance, and for example, it may be selected from the group consisting of DNA, RNA, polypeptides, proteins, ligands, enzymes, antibodies, antigens, natural compounds, synthetic compounds and bioactive molecules, but the present disclosure is not limited thereto. According to an exemplary embodiment of the present disclosure, the deletion of arginase-1 gene may be induced by the cre-loxp system, but the present disclosure is not limited thereto.

In the present disclosure, arginase-1 protein may be expressed in cells, and arginase-1 protein may be expressed in immune cells expressing Iba-1. In addition, the Iba-1 protein is expressed in both of macrophages and microglia, and among these, arginase-1 protein, which is expressed in macrophages rather than microglia, is involved in ischemic cerebrovascular disease such as cerebral infarction. According to an exemplary embodiment of the present disclosure, the arginase-1 inhibitor in the present disclosure may inhibit the activity of arginase-1 protein in macrophages, and the macrophages may be infiltrating macrophages, but the present disclosure is not limited thereto.

In the present disclosure, ischemic cerebrovascular disease refers to a disease in which various types of pathological abnormalities occur in blood vessels supplying blood to the brain, resulting in the disruption of normal cerebral blood flow, and it may be described interchangeably with the terms ischemic stroke or cerebral infarction.

Ischemia in the present disclosure refers to a condition in which blood supply is blocked and insufficient, causing necrosis in the relevant tissue area. By using the composition of the present disclosure, it is possible to recover damage to the brain ischemic area and treat ischemic cerebrovascular disease.

The pharmaceutical composition of the present disclosure may be applied in any dosage form, and more specifically, it may be formulated and used in oral dosage forms and parenteral dosage forms such as topical preparations, suppositories and sterilized injection solutions according to conventional methods.

Among the oral dosage forms, the solid dosage form is in the form of tablets, pills, powders, granules, capsules and the like, and it may be prepared by mixing at least one excipient, such as starch, calcium carbonate, sucrose, lactose, sorbitol, mannitol, cellulose, gelatin and the like, and in addition to simple excipients, it may include a lubricant such as magnesium stearate and talc. Further, in the case of a capsule dosage form, it may further include a liquid carrier such as fatty oil, in addition to the above-mentioned substances.

Among the oral dosage forms, liquid dosage forms include suspensions, oral solutions, emulsions, syrups and the like, and they may include various excipients such as wetting agents, sweeteners, fragrances, preservatives and the like, in addition to water and liquid paraffin, which are commonly used simple diluents.

The parenteral dosage form may include sterilized aqueous solutions, non-aqueous solvents, suspensions, emulsions, freeze-dried preparations and suppositories. Non-aqueous solvents and suspensions may include propylene glycol, polyethylene glycol, vegetable oil such as olive oil and injectable ester such as ethyl oleate. As a base for suppositories, witepsol, macrogol, Tween 61, cacao, laurin, glycerogenatin and the like may be used. Without being limited thereto, any suitable agent known in the art may be used.

In addition, the pharmaceutical composition of the present disclosure may be used alone, or it may be used in combination with existing agents or methods for preventing and/or treating ischemic cerebrovascular disease, and in this case, it may be administered simultaneously or sequentially.

The pharmaceutical composition of the present disclosure may be administered with a pharmaceutically acceptable carrier, and when it is administered orally, binders, lubricants, disintegrants, excipients, solubilizers, dispersants, stabilizers, suspending agents, colorants, flavors and the like may be used, and in the case of injections, buffers, preservatives, analgesics, solubilizers, isotonic agents, stabilizers and the like may be mixed and used, and in the case of topical administration, bases, excipients, lubricants, preservatives and the like may be used.

The pharmaceutical composition of the present disclosure may be formulated in a suitable form according to methods known in the art along with a pharmaceutically acceptable carrier as described above.

In the pharmaceutical composition of the present disclosure, the pharmaceutical composition may be administered in a pharmaceutically effective amount.

A pharmaceutically effective amount in the present disclosure means an amount sufficient to treat a disease with a reasonable benefit/risk ratio that is applicable to medical treatment and does not cause side effects.

The effective dosage level of the pharmaceutical composition may be variously determined by elements including the purpose of use, the patient's age, gender, weight and health status, type and severity of the disease, activity of the drug, sensitivity to the drug, method of administration, time of administration, route of administration and excretion rate, treatment period, combination or drugs used simultaneously, and other factors well known in the medical field.

For example, although it is not constant, it may be administered generally at 0.001 to 100 mg/kg, and preferably, at 0.01 to 10 mg/kg once or several times a day. The above dosage does not limit the scope of the present disclosure in any way.

The pharmaceutical composition of the present disclosure may be administered to any animal that can develop ischemic cerebrovascular disease, and the animals may include, for example, humans and primates as well as livestock such as cows, pigs, horses and dogs.

The pharmaceutical composition of the present disclosure may be administered through an appropriate administration route depending on the formulation type, and may be administered through various oral or parenteral routes as long as it can reach the target tissue. The method of administration does not need to be particularly limited, and may be administered by conventional methods such as oral, rectal or intravenous, intramuscular, dermal application, intrarespiratory inhalation, intrauterine, dural or intra-cerebroventricular injection and the like.

In addition, the present disclosure provides a method for screening a substance for preventing or treating ischemic cerebrovascular disease, including the steps of (a) treating stroke-inducing mice with a test substance to induce the deletion of a gene encoding the arginase-1 protein or the inhibition of arginase-1 protein function: (b) measuring the inflammatory cytokine expression rate in stroke-induced mice in which a gene in the arginase-1 protein is defective or the function of the arginase-1 protein is inhibited; and (c) selecting a candidate substance that reduces the expression rate of inflammatory cytokines in stroke-induced mice that are treated with the test substance compared to stroke-induced mice that are not treated with the test substance.

The method for screening a substance for preventing or treating ischemic cerebrovascular disease according to the present disclosure will be described step by step.

Step (a): Treating stroke-inducing mice with a test substance to induce the deletion of a gene encoding the arginase-1 protein or the inhibition of arginase-1 protein function.

The test substance is any substance that can cause defects in the expression of arginase-1 gene or inhibit the function of arginase-1 protein, and is not limited to the type or kind of substance, and for example, it may be selected from the group consisting of DNA, RNA, polypeptides, proteins, ligands, enzymes, antibodies, antigens, natural compounds, synthetic compounds and bioactive molecules, but the present disclosure is not limited thereto.

Step (b): Measuring the inflammatory cytokine expression rate in stroke-induced mice in which a gene in the arginase-1 protein is defective or the function of the arginase-1 protein is inhibited.

The method of measuring the expression rate of the inflammatory cytokine may use any method known in the art, and for example, it may be detected by using spectroscopic, photochemical, biochemical, immunochemical, electrical, absorbance, chemical and other methods. More specifically, it may include ELISA, RIA, Western blot, PCR, immunoprecipitation, immunostaining, flow cytometry/FACS, chromatography and the like, but the present disclosure is not limited thereto.

Step (c): Selecting a candidate substance that reduces the expression rate of inflammatory cytokines in stroke-induced mice that are treated with the test substance compared to stroke-induced mice that are not treated with the test substance.

In step (c), based on the results in step (b), if the expression rate of inflammatory cytokines in stroke-induced mice that are treated with the test substance is reduced compared to stroke-induced mice that are not treated with the test substance, the test substance is selected as a substance for preventing or treating ischemic cerebrovascular disease.

The inflammatory cytokines may be measured in macrophages, and according to an exemplary embodiment of the present disclosure, the inflammatory cytokines may be measured in microglia, but the present disclosure is not limited thereto.

The inflammatory cytokines may be Gdf9, Gdf15 and Tgfb1, which are inflammatory signaling molecules that mediate TGF-beta signaling, and they may be cytokines such as Interleukin-6, oncostatin M, Interleukin-16 and interleukin-1 beta, which are well known to induce inflammation, but the present disclosure is not limited thereto.

The screening method is a method of comparing an increase or decrease in the expression or activity of the arginase-1 inhibitor or a gene thereof in the presence or absence of a candidate substance for a therapeutic agent of ischemic cerebrovascular disease, and it may be advantageously used to screen for arginase-1 inhibitors or agonists of a gene thereof, improvement or treatment of cardiovascular diseases and the like.

Since the corresponding features can be replaced in the above-mentioned part, the descriptions thereof are omitted.

Hereinafter, the present disclosure will be described in detail through examples to aid understanding. However, the following examples only illustrate the content of the present disclosure, and the scope of the present disclosure is not limited to the following examples. Examples of the present disclosure are provided to more completely explain the present disclosure to those skilled in the art.

### EXAMPLE

### Materials and Methods

### 1. Experimental Animals

For the animals for cell-specific fluorescent labeling, animals that were born from a cross between B6.129P2(Cg)-Cx3cr1tm1Litt/J (Cx3cr1 GFP, Jaxon Laboratory, Bar Harbor, ME USA) mice and B6.129X1-Gt(ROSA)26Sortm1(EYFP)Cos/J (Rosa-stop-e YFP, Jaxon Laboratory, Bar Harbor, ME USA) and B6.129P2-Lyz2tm1(cre)Ifo/J (LysM-cre, Jaxon Laboratory, Bar Harbor, ME USA) were used. For cell-specific arginase-1 gene deletion and functional tests, animals that were born from a cross between C57BL/6-Arg1tm1Pmu/J (Arg1 floxed, Jaxon Laboratory, Bar Harbor, ME USA) mice and B6.129P2-Lyz2tm1(cre)Ifo/J (Jaxon Laboratory, Bar Harbor, ME USA) were used. For the crossbred animals, animals that were simultaneously confirmed to be homozygous for the Arg1 floxed sequence and heterozygous for the LysM cre sequence were used through genotyping. As the control group for the arginase-1 gene function tests, C57BL/6-Arg1tm1Pmu/J (Arg1 floxed) mice were used. Animals were raised in individually ventilated cages prior to euthanasia, and all animal experiment methods were approved by the Laboratory Animal Care Committee of Ajou University.

### 2. Photothrombotic Ischemic Stroke Model

The photothrombotic ischemic stroke model was performed in adult mice after anesthetizing them with ketamine. Anesthetized animals were fixed in a stereotaxic frame on a heating pad at 37.0° C., and two skin incisions were made. Afterwards, rose bengal (10 μg/mL, Sigma, St. Louis, USA) was administered (100 μL/g, i.p) and waited for 5 minutes to allow the drug to be absorbed into the bloodstream. After 5 minutes, a light source was irradiated onto the skull for 25 minutes. For the light source, an illuminator (Thermo Scientifics, Massachusetts, USA) using a 150w halogen lamp (Phillips, Eindhoven, Netherlands) was used. The light source was illuminated through an aperture with a diameter of 1.5 mm on the skull 1.8 mm lateral from bregma. In the case of animals undergoing behavioral experiments, the light source was irradiated in a direction opposite to the direction of the dominant upper limb, and for all other animals, the light source was irradiated on the right hemisphere of the brain. After the surgery was completed, the cranial skin tissue was sutured, and povidin was applied to the animal.

### 3. Behavioral Experiment to Evaluate Motor Function

All behavioral experiments were conducted with restricted feed. The restricted amount of feed was given once a day in an amount equivalent to 10% of the animal's body weight after measuring the body weight. As behavioral experiments to evaluate upper limb motor function, a pellet test, a ladder walking test, a cylinder test and a neurological severity score (mNSS) were conducted. All behavioral experiments were conducted at the same time. In order to motivate the experimental animals, sucrose pellets (20 mg, Bioserv, New Jersey, USA) equivalent to 10% of the daily feed were fed during the first 2 days of food restriction. In subsequent experiments, animals that did not consume pellets or did not use their upper limbs were excluded from the experiment. Animals that showed a weight loss of more than 20% during the feed restriction period were given an additional 20% of the daily feed.

The pellet experiment was conducted by inducing the experimental animals to ingest the pellets using their upper extremities within an apparatus with a narrow opening 0.5 mm wide. In the case of the pellet experiment, training was conducted for 8 days after a 2-day adaptation period. The adaptation period was 2 days, with 20 minutes of group adaptation training and 20 minutes of individual adaptation training. On the first day of training, pellets were separated into left-handed and right-handed animals in an apparatus with a single narrow opening. In subsequent training, the number of pellets consumed using the dominant upper extremity was measured within a box with two narrow openings.

The ladder walking experiment was conducted on an apparatus with 60 cm long ladders that were spaced at 2 cm intervals. In the case of the ladder walking experiment, training was conducted for 3 days after a 2-day adaptation period. During the two-day adaptation period, the animals underwent adaptation training through voluntary ladder walking for a total of five times. After adaptation training, the ladder walking experiment measured the rate of left and right upper extremity falls in five attempts to cross the ladder.

In the case of the cylinder test, the animal's ability to perform behavioral tests decreased with repeated experiments, and thus, it was conducted without an adaptation period. In the cylinder test, the animal was placed in a cylindrical device with a height of 17.5 cm and a diameter of 8.8 cm, and the number of times the upper limb touched the wall of the cylinder was measured.

Neurological severity evaluation recorded scores by comprehensively evaluating the folding of the upper and lower extremities, cranial rotation rate, walking on level ground, cognition and proprioception, balance beam walking, auditory response, auricular stimulation, corneal stimulation, seizure presence and the like.

For all behavioral experiments, the final measurement of training was set as the baseline. After completing the training, photothrombotic ischemic stroke was induced in the animals, and all behavioral tests were repeated at one-week intervals for four weeks to measure motor function. All behavioral experiments were recorded on video (GoPro, California, USA) and analyzed at 0.2× speed after conducting the behavioral experiments.

### 4. Cytokine Polymerase Chain Reaction (PCR) Analysis

For cytokine PCR analysis, tissue samples from animals that had undergone a 7-day recovery period after ischemic stroke were used. The cerebrum was isolated from animals euthanized with CO₂. The separated cerebral tissue was cut into 3×3 mm pieces, and tissue surrounding the stroke was collected. The collected tissues were separated at the single cell level following the protocol of the Adult brain dissociate kit (Miltenyi, North Rhine-Westphalia, Germany). The separated samples were labeled with Anti-CD11b (Alexa488, Biorad, California, USA) and Anti-CD45 (Alexa594, Biorad, California, USA) fluorescent antibodies (10 ul/1.0×10\hat{ }5 cells) at 4° C. for 1 hour. The labeled cell sample was divided into two samples, macrophages and microglia, through an automated cell sorter (FACSAria III, Becton Dickinson and Company (BD), New Jersey, USA). Each sample was used for RNA extraction after centrifugation. RNA extracted from cells was synthesized into cDNA (RT2 First strand kit, Qiagen, Düsseldorf, Germany), and then, samples were prepared by using the RT2 profiler PCR array kit (Qiagen, Düsseldorf, Germany). The prepared samples were quantitatively analyzed by using the Quant3 (Applied Biosystems, Massachusetts, USA) program.

5. RNA Extraction, cDNA Synthesis and Reverse Transcription Polymerase Chain Reaction (PCR)

The prepared cell and tissue samples were treated with Qiazol (Qiagen, Düsseldorf, Germany) at a rate of 10 μL/mg to lyse the cells. Afterwards, 20% of the Qiazol volume of chloroform was mixed and centrifuged at 15,000 g at 4° C. for 15 minutes. Afterwards, the supernatant was separated, treated with equal amounts of isopropane alcohol and yeast tRNA (20 μg/ml) and incubated at −20° C. for 1 hour. The cultured sample was centrifuged at 15,000 g at 4° C. for 30 minutes to pellet the eluted RNA. The RNA pellet was washed with 70% ethanol solution and centrifuged at 15,000 g at 4° C. for 5 minutes. After centrifugation, ethanol was removed, and it was dried at room temperature for 5 minutes. The dried RNA was diluted in RNase free water and stored at −70° C. RNA was synthesized into cDNA by using cDNA synthesis mater mix (Cell Safe, Korea).

Reverse transcription polymerase chain reaction (PCR) experiments were amplified by adding complementary primers and PCR mixture (Bioneer, Korea) to the synthesized cDNA sample. In this experiment, a primer complementary to the sequence below was used. Meanwhile, sequence information for the primers and Cre protein used in the examples is shown in Table 1 below.

TABLE 1

| | |
|---|---|
| Cre recombinase | MSNLLTVHQNLPALPVDATSDEVRKNLMDM |
| | FRDRQAFSEHTWKMLLSVCRSWAAWCKLNN |
| | RKWFPAEPEDVRDYLLYLQARGLAVKTIQQ |
| | HLGQLNMLHRRSGLPRPSDSNAVSLVMRRI |
| | RKENVDAGERAKQALAFERTDFDQVRSLME |
| | NSDRCQDIRNLAFLGIAYNTLLRIAEIARI |
| | RVKDISRTDGGRMLIHIGRTKTLVSTAGVE |
| | KALSLGVTKLVERWISVSGVADDPNNYLFC |
| | RVRKNGVAAPSATSQLSTRALEGIFEATHR |
| | LIYGAKDDSGQRYLAWSGHSARVGAARDMA |
| | RAGVSIPEIMQAGGWTNVNIVMNYIRNLDS |
| | ETGAMVRLLEDGD |
| | (SEQ ID NO: 1) |
| Arg1 | Forward: |
| | 5′-AGGAGCTGTCATTAGGGACATC-3′ |
| | (SEQ ID NO: 2) |
| | Reverse: |
| | 5′-CTCCAAGCCAAAGTCCTTAGAG-3′ |
| | (SEQ ID NO: 3) |
| CR3 | Forward: |
| | 5′-AAGCAGCTGAATGGGAGGAC-3′ |
| | (SEQ ID NO: 4) |
| | Reverse: |
| | 5′-TAGATGCGATGGTGTCGAGC-3′ |
| | (SEQ ID NO: 5) |
| Galectin-3 | Forward: |
| | 5′-CTACCCAGGGGCTGCTTATC-3′ |
| | (SEQ ID NO: 6) |
| | Reverse: |
| | 5′-AGCGGGGGTTAAAGTGGAAG-3′ |
| | (SEQ ID NO: 7) |
| CD36 | Forward: |
| | 5′-TGGAGGCATTCTCATGCCAG-3′ |
| | (SEQ ID NO: 8) |
| | Reverse: |
| | 5′-AAGACACAGTGTGGTCCTCG-3′ |
| | (SEQ ID NO: 9) |
| Cd68 | Forward: |
| | 5′-GGGCTCTTGGGAACTACACG-3′ |
| | (SEQ ID NO: 10) |
| | Reverse: |
| | 5′-AGACTGTACTCGGGCTCTGA-3′ |
| | (SEQ ID NO: 11) |
| TREM2 | Forward: |
| | 5′-CTTGCTGGAACCGTCACCAT-3′ |
| | (SEQ ID NO: 12) |
| | Reverse: |
| | 5′-CTCTTGATTCCTGGAGGTGC-3′ |
| | (SEQ ID NO: 13) |
| 18s | Forward: |
| | 5′-CGCGGTTCTATTTTGTTGGT-3′ |
| | (SEQ ID NO: 14) |
| | Reverse: |
| | 5′-AGTCGGCATCGTTTATGGTC-3′ |
| | (SEQ ID NO: 15) |

The amplification process was performed in 34 cycles: 94° C. for 30 seconds, 55-65° C. for 30 seconds and 72° C. for 30 seconds. The amplified sample was subjected to electrophoresis on a 1.0% agarose gel to detect the amplified DNA, and PCR for quantitative analysis was analyzed by using the ABM7500 (Applied Biosystems, Massachusetts, USA) software.

### 6. Tissue Immunostaining and Image Taking

Tissue samples for tissue immunostaining were obtained from fixed tissues through perfusion with PBS and 4% PFA after anesthetizing the experimental animals with ketamine. After perfusion, tissues that were collected from the experimental animals were treated with 10% sucrose solution and 30% sucrose solution at 4° C. The sucrose-treated tissues were sectioned at 30 μm thickness by using a frozen microtome. The sectioned tissue was attached to a tissue slide, and tissue immunostaining was performed. Tissue slides were incubated at room temperature for 30 minutes in a solution including 0.3% triton x-100 and 10% NGS before immunostaining. For the primary antibodies for tissue immunostaining, anti-Arg1 c-term (ab60176, Abcam, 1:500), anti-GFAP (Dako, 033401, 1:1,000), anti-Iba1 (Wako, 019-19741, 1:500), anti-MAP2 (Chemicon, ab5622, 1:500), anti-fibronectin (Merck, mab1940, 1:500), biotinylated-WFA(Vector Lab, 1355-21:100, 15 μg/mL) anti-vglut2 (Synaptic Systems, 135416, 1:500) and anti-PSD95 (Invitrogen, 51-6900, 1:200) antibodies were used. The antibody labeling process was carried out by incubating the primary antibody that was diluted in 10% NGS on the tissue slide for 16 hours at 4° C., followed by incubating the secondary antibody for 2 hours at room temperature. Tissue slides after antibody incubation were fixed with mounting medium and photographed under a microscope. Stained tissue slides were photographed by using a Zeiss LSM880 (Jena, Germany) microscope. For synapse imaging, tissue slides that were stained with vglut2 and PSD95 were used. Synapse images were taken and analyzed by using a Nikon AIR (Tokyo, Japan) microscope. Each tissue slide was photographed at 2× magnification with a 100× lens, and 3.75 μm z-stack images were taken at 0.75 μm intervals.

### 7. Primary Culture of Peritoneal Macrophages

After euthanizing the mouse with CO₂, 10 mL of cold PBS was injected intraperitoneally. The mouse was massaged in the abdomen for 1 minute to remove the peritoneal lavage fluid, and it was centrifuged at 300 g at 4° C. to collect the cells. The collected cells were dissolved in DMEM (10% FBS, Hyclone, Washington, D.C., USA) and seeded in a culture dish. The dispensed cells were cultured in a 37° C. incubator for 24 hours and then used for RNA extraction.

### 8. Western Blot

Tissues collected for protein extraction were rapidly frozen and dissociated in a lysis reagent by using a homogenizer. Afterwards, it was centrifuged at 15,000 g for 5 minutes at 4° C. to separate only the supernatant, and it was cultured at 95° C. for 5 minutes. The tissue samples were loaded onto Mini-PROTEAN® TGX™ Gel (Biorad, California, USA) and subjected to electrophoresis at 100 V for 1 hour and 30 minutes. After electrophoresis, the gel was transferred to a PVDF membrane at 275 mA for 1 hour and 30 minutes. After transfer, the PVDF membrane was incubated with anti-Arg1 primary antibody (ab60176, Abcam, 1: 500) that was diluted in 5% skim milk for 16 hours at 4° C. Next, it was incubated with HRP conjugated anti-goat igG (7074S, cell signaling, 1: 5,000) secondary antibody at room temperature for 2 hours and then detected with x-ray film.

### <Example 1> Confirmation of the Expression of Arginase-1 in Photothrombotic Ischemic Stroke Model

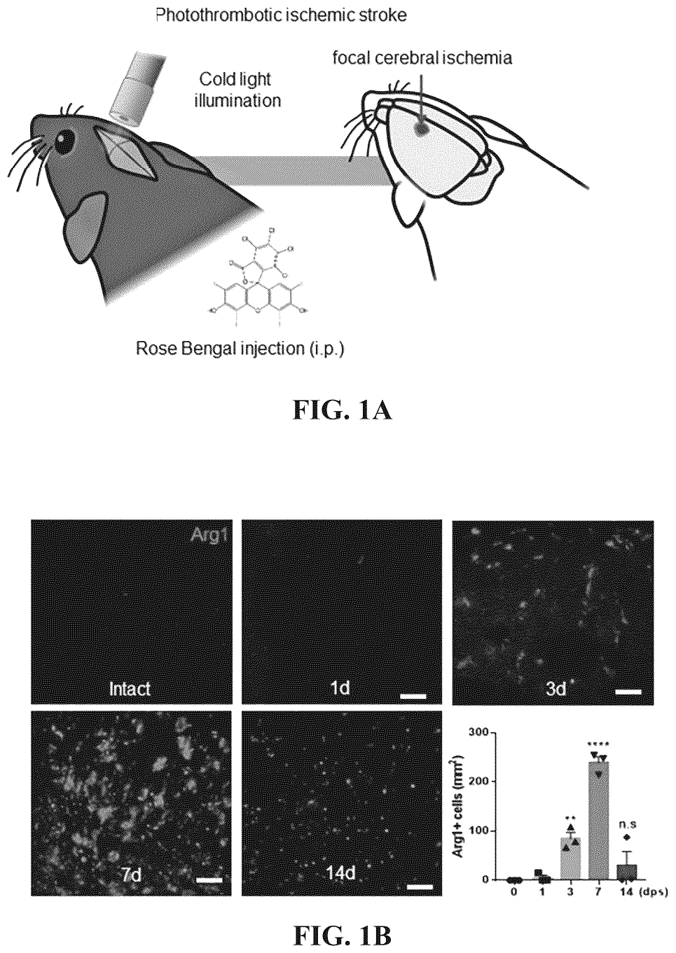

In order to confirm the expression pattern of arginase-1 protein in the photothrombotic stroke model, local thrombosis was induced in the cerebral cortex of mice, and a stroke model was constructed. The photothrombotic stroke model can be induced by using the drug Rose bengal (FIG. 1A). After the administration of rose bengal, a light source was irradiated to induce a stroke in the relevant area. Animals were euthanized 1, 3, 7 and 14 days after stroke induction, and the expression pattern of arginase-1 protein was confirmed through tissue immunostaining.

The arginase-1 protein was confirmed to be expressed locally within the lesion, increasing until day 7, and then decreasing again (FIG. 1B).

### <Example 2> Identification of Cell Types Expressing Arginase-1 Protein after Stroke

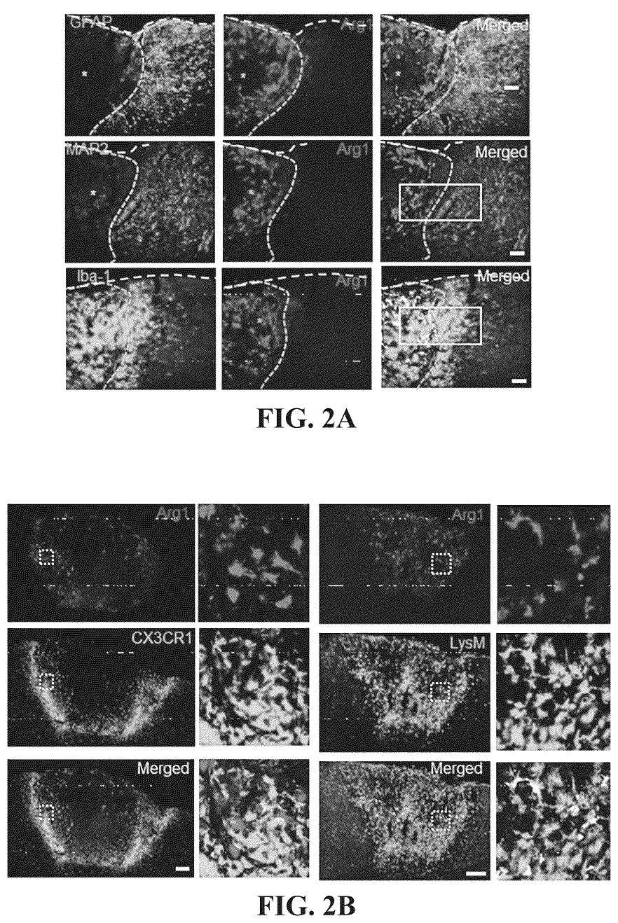

In order to identify cells expressing arginase-1 protein in the stroke model, cells expressing arginase-1 were identified through tissue immunostaining on day 7, when the expression of arginase-1 was most active. In tissue immunostaining, as result of staining with an antibody that can label neurons (Map2), astrocytes and immune cells (Iba-1), it was confirmed that most of the arginase-1 protein was expressed in immune cells expressing Iba-1. Since the Iba-1 protein is expressed in both of infiltrating macrophages and microglia, it is difficult to distinguish between the two cell types (FIG. 2A). In order to distinguish between the two cells, animals were used to label specific cells with fluorescence.

As a result of the experiment, it was confirmed that the arginase-1 protein was expressed through LysM-eYFP fluorescence, which labels infiltrating macrophages, rather than CX3CR1 GFP fluorescence, which labels microglia. Therefore, this suggests that infiltrating macrophages express arginase-1 protein after stroke (FIG. 2B).

### <Example 3> Verification of the Inhibition of Expression of Arginase-1 Protein Specific to Infiltrating Macrophages

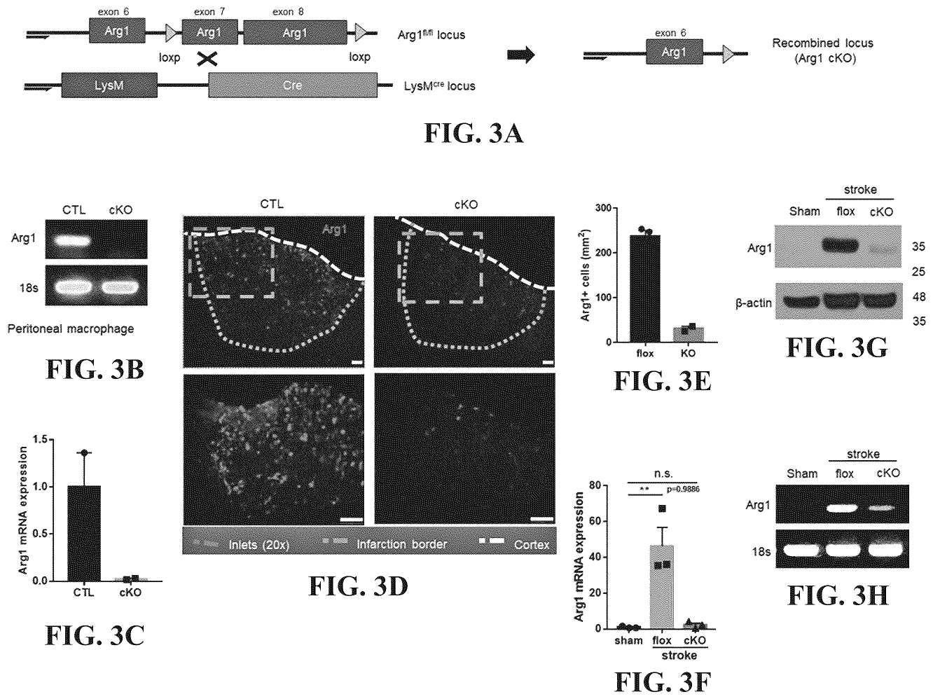

Through fluorescence-labeled infiltrating macrophage animals, it was confirmed that the arginase-1 protein is expressed in infiltrating macrophages after stroke. The cre-loxp system was used to specifically suppress the expression of arginase-1 protein in infiltrating macrophages. The loxp sequence which is inserted into the upper part of exon 7 and the lower part of exon 8 of the arginase-1 gene is a site where gene deletion can be induced by the cre protein. Therefore, in order to confirm the inhibition of the expression of arginase-1 protein due to a gene deletion between the two loxp sequences that are present in the arginase-1 gene, by crossing arginase-1 floxed animals with animals expressing the cre protein from the LysM promoter, the arginase-1 gene was induced to be specifically depleted in infiltrating macrophages expressing LysM (FIG. 3A).

First of all, by confirming the expression of Arg1 in peritoneal macrophages, which are well known to express LysM, it was confirmed that the expression of arginase-1 was suppressed in the corresponding animals (FIGS. 3B and 3C). Next, in order to confirm whether the expression of arginase-1 was well suppressed in the experimental animal tissue, photothrombotic stroke was induced, and then, the expression levels of protein and RNA in the lesion tissue were determined through tissue immunostaining (FIGS. 3D and 3E), PCR experiments. (FIG. 3F and FIG. 3H) and Western blot experiment (FIG. 3G). As a result of the experiments, it was verified that the expressions of arginase-1 protein and genome was suppressed by more than 95% 7 days after photothrombotic stroke in the animal.

### <Example 4> Behavioral Experiment for Evaluating Functional Recovery after Stroke: mNSS Scoring

Neurological severity assessment was performed to test whether the inhibition of arginase-1 expressed by infiltrating macrophages after stroke contributes to motor function recovery. The experiment induced ischemic stroke in arginase-1-deficient animals and was repeated at one-week intervals for a total of four weeks to evaluate the recovery of motor function.

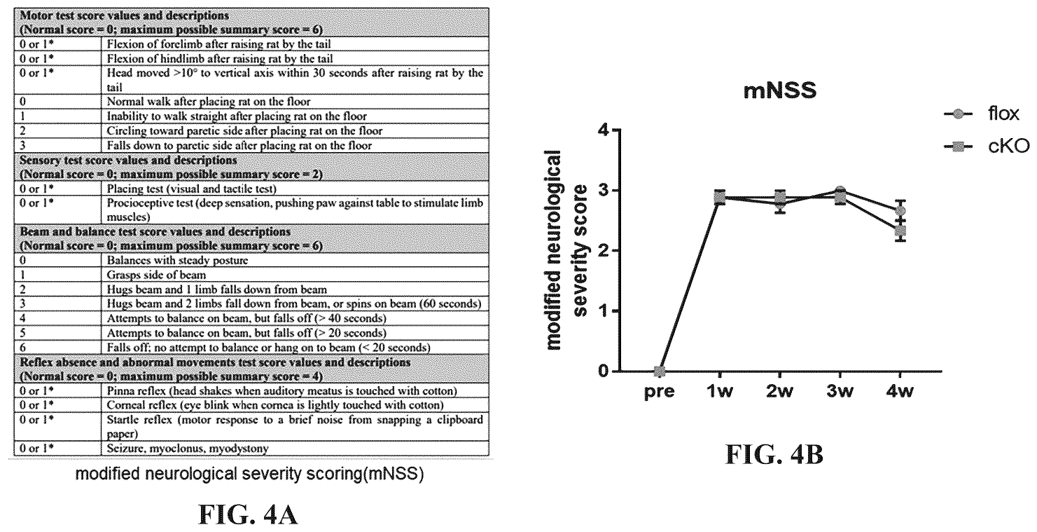

As a result of the experiment, the motor function evaluation (folding of the upper and lower limbs and skull rotation rate) that appears when the animal is lifted by the tail at the current stroke level was impaired, but the proprioception evaluation, column walking balance evaluation, sensory function evaluation and reflex function evaluation appeared to be at normal levels (FIG. 4A). In addition, no statistically significant recovery of behavioral function was observed in arginase-1-deficient animals during the 4-week evaluation. This result is inferred because the stroke was induced in a local area associated with upper limb motor function (FIG. 4B).

### <Example 5> Behavioral Experiment for Evaluating Functional Recovery after Stroke: Pellet Experiment

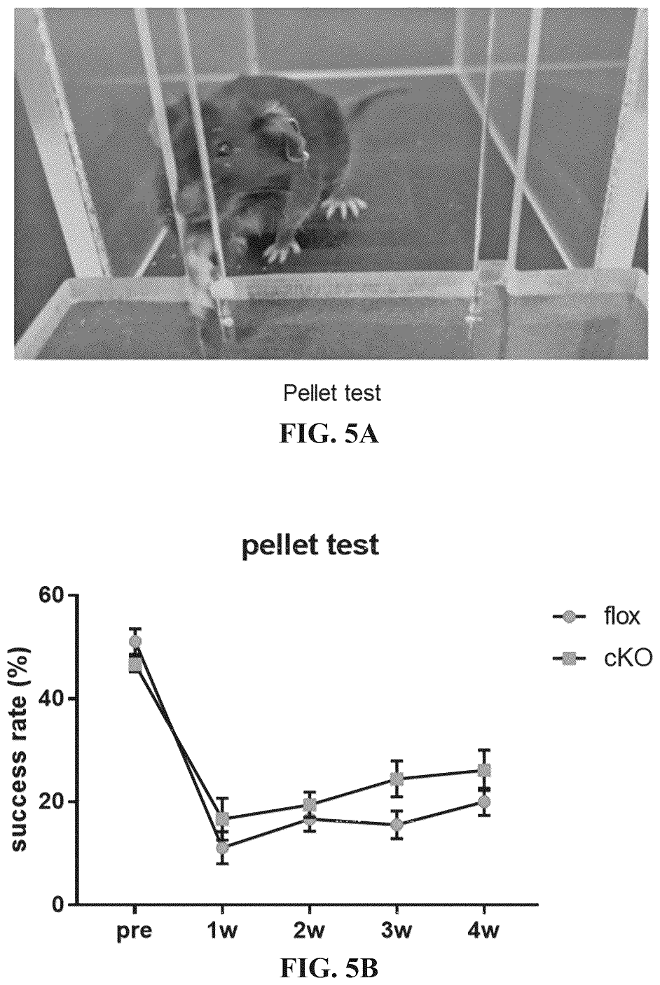

The following behavioral experiment is an experiment for evaluating motor function through pellet ingestion by using the upper extremities in trained animals. By limiting the amount of feed the animal receives to encourage it to consume pellets as a reward and then placing the pellet across a narrow gap, it is possible to evaluate the upper limb function when taking the pellet. Before stroke induction, left-handed and right-handed animals were distinguished during a training period of about 2 weeks, and then, a stroke was induced in the cerebral cortex in a direction opposite to the direction of the dominant upper limb (FIG. 5A).

In this experiment, which was conducted for 4 weeks at 1-week intervals after stroke induction, although it was not statistically significant, the recovery of motor function after stroke was generally improved in arginase-1-deficient animals (FIG. 5B).

### <Example 6> Behavioral Experiment for Evaluating Functional Recovery after Stroke: Cylinder Experiment

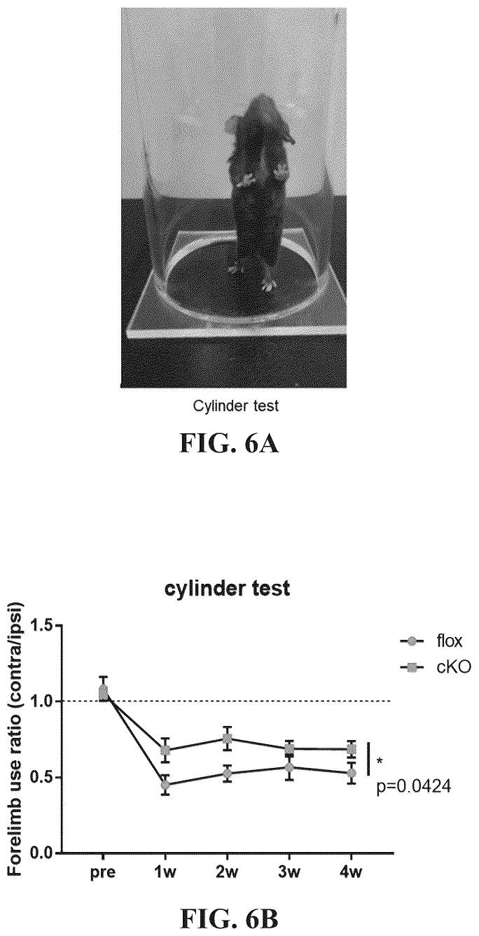

Within cylindrical structures, mice show an instinctive behavior to explore upward. During this process, the mouse stands up by using its upper limbs. At this time, functional impairment can be evaluated through the wall contact ratio of the left and right upper limbs. Since the upper extremity on the dysfunctional side had a reduced number of wall contacts, upper extremity function was evaluated by measuring the ratio (FIG. 6A).

As a result, it was confirmed that motor function was recovered statistically significantly in arginase-1-deficient animals (FIG. 6B).

### <Example 7> Behavioral Experiment for Evaluating Functional Recovery after Stroke: Ladder Walking Experiment

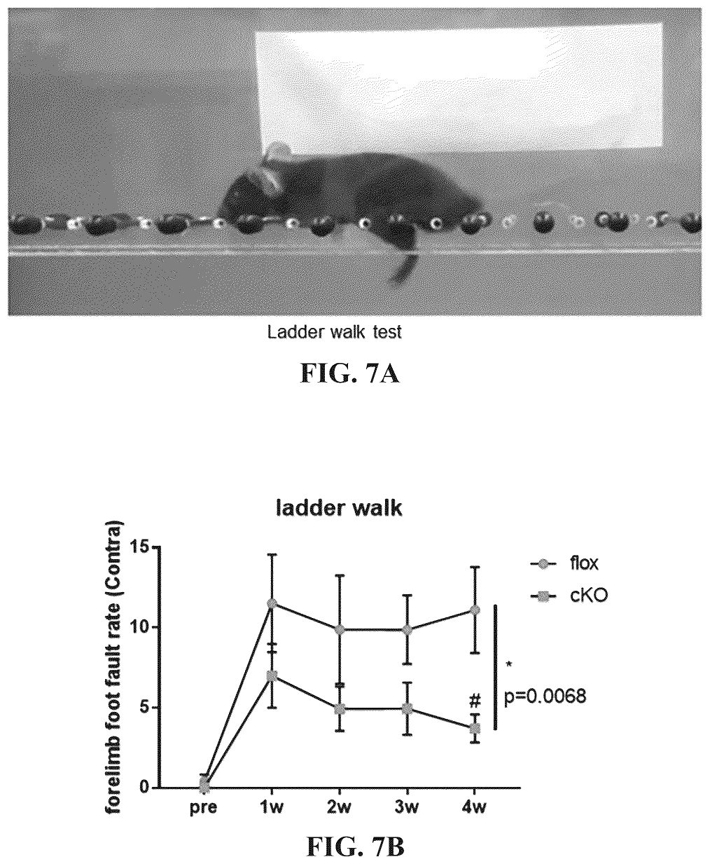

The ladder walking experiment is an experiment that can evaluate motor function during walking by using animals that are trained to walk on ladders placed at regular intervals. If the motor function of an animal trained to cross a ladder is impaired, the motor function can be evaluated by measuring the number of times the leg falls between the ladders while walking (FIG. 7A).

In this experiment, statistically significant recovery of motor function was confirmed in arginase-1 deficient animals. In particular, the most statistically significant difference was confirmed in the walking experiment conducted at Week 4. By including the behavioral experiments described above, it was finally possible to experimentally prove that the arginase-1 protein of infiltrating macrophages had a negative effect on the recovery of motor function after stroke (FIG. 7B).

### <Example 8> Genetic Phenotypic Changes in Microglia Around Lesions after Stroke in Arginase-1 Gene Deficient Animals

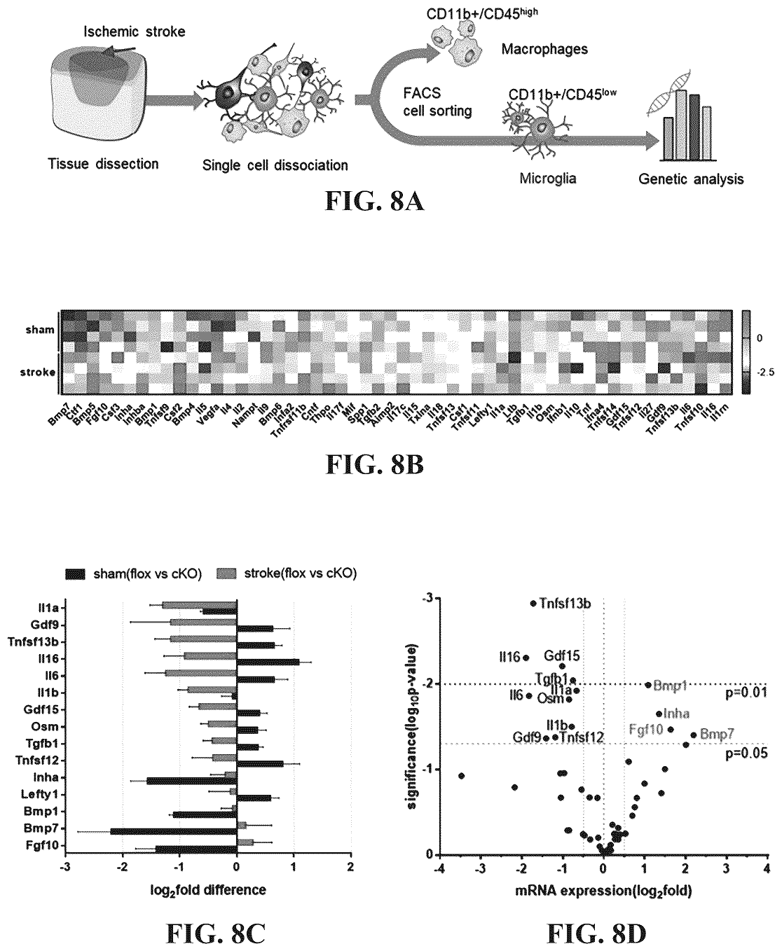

After stroke, infiltrating macrophages expressing arginase-1 are localized in the lesion area where they cannot function normally due to neuronal cell death. In order to recover function after a stroke, normally functioning neurons must influence the living lesion periphery. Therefore, in the interaction between infiltrating macrophages and microglia after stroke, arginase-1 deficiency may affect microglia and the inflammatory environment around the lesion. For this reason, microglia cells were isolated from surrounding tissues after stroke in arginase-1 deficient animals and tested for changes in the expression of inflammatory signaling factors. In the experiment, photothrombotic stroke was induced in arginase-1 deficient animals, and tissue surrounding the lesion was obtained one week later. The tissue was separated at the single cell level, and only microglia were isolated through FACS experiment. The isolated microglia were measured for changes in the level of inflammatory signal molecules through a PCR array experiment (FIG. 8A).

In arginase-1-deficient animals, microglia after stroke had a significant decrease in inflammatory signaling molecules (Gdf9, Gdf15, Tgfb1) that mediate TGF-beta signaling, and it was confirmed that the levels of cytokines including Interleukin-6, oncostatin M, Interleukin-16 and interleukin-1 beta, which are well known to induce inflammation, were significantly reduced. In addition, after tissue damage, it was confirmed that the expression levels of the fibrosis suppressing genes BMP1, BMP7 and fgf10 significantly increased, while the tissue fibrosis promoting genes tnfsf13b, tnsf12 and the like decreased (FIGS. 8B to 8C).

These changes are not only due to a decrease in inflammatory signaling factors that have a negative impact on tissue recovery, but also because arginase-1 gene deletion in macrophages affects the cell phenotypic changes in microglia around the lesion, which indirectly shows an interaction between the two cells.

### <Example 9> Confirmation of Pathological Changes in Tissue Surrounding the Lesion after Stroke

As confirmed in the previous experiments, arginase-1 gene deletion in infiltrating macrophages shows a decrease in TGF-beta signaling and fibrosis-related genes in microglia around the lesion. Therefore, histological changes were confirmed in arginase-1 gene deficient animals. Tissue fibrosis, which is represented by fibronectin, can be observed in brain tissue after stroke. This tissue fibrosis is accompanied by the deposition of chondroitin sulfate proteoglycan (CSPG; CS-56), which inhibits synapse formation and axon growth.

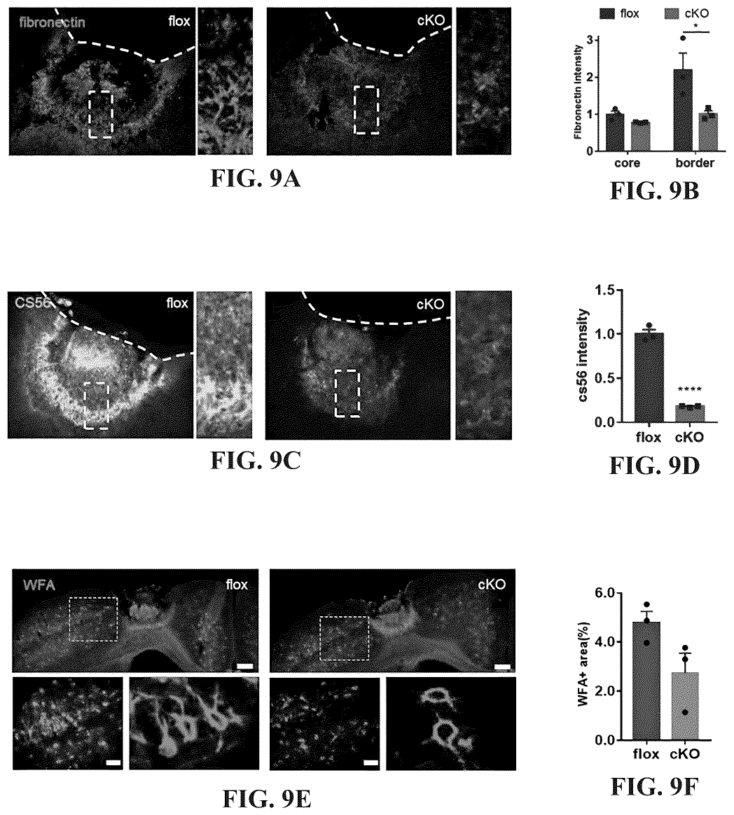

In tissue immunostaining experiments, it was confirmed that tissue fibrosis and CSPG deposition after stroke were significantly reduced in arginase-1 deficient animals (FIGS. 9A to 9D). This result corresponds to changes in gene expression of microglia cells around lesions in arginase-1 deficient animals in the previous experiment. In addition, it was confirmed that peri-neuronal net (WFA), which is a central nervous system neuron structure, was decreased in arginase-1 deficient animals (FIGS. 9E and 9F). The peri-neuronal net is a complex of CSPGs and is well known to inhibit synapse formation in central nervous system neurons.

These results suggest that the expression of arginase-1 in infiltrating macrophages induced fibrosis and deposition of regenerative inhibitors in the tissue surrounding the lesion through interaction with microglia.

### <Example 10> Experiment on Synaptic Structure and Microglia Phagocytic Activity in the Periphery of Stroke

Recent studies have largely covered the fact that the phagocytosis of microglia cells is involved in synaptic regulation in stroke and neurodegenerative diseases. As revealed in the previous experiments, in order to confirm whether the expression of arginase-1 in infiltrating macrophages affected the phenotypic changes with microglia, particularly the phagocytosis of microglia, the expression of Trem2, Galactin-3, CD36, CD68 and CR3, which are well known as microglia phagocytosis genes, was confirmed through PCR experiments.

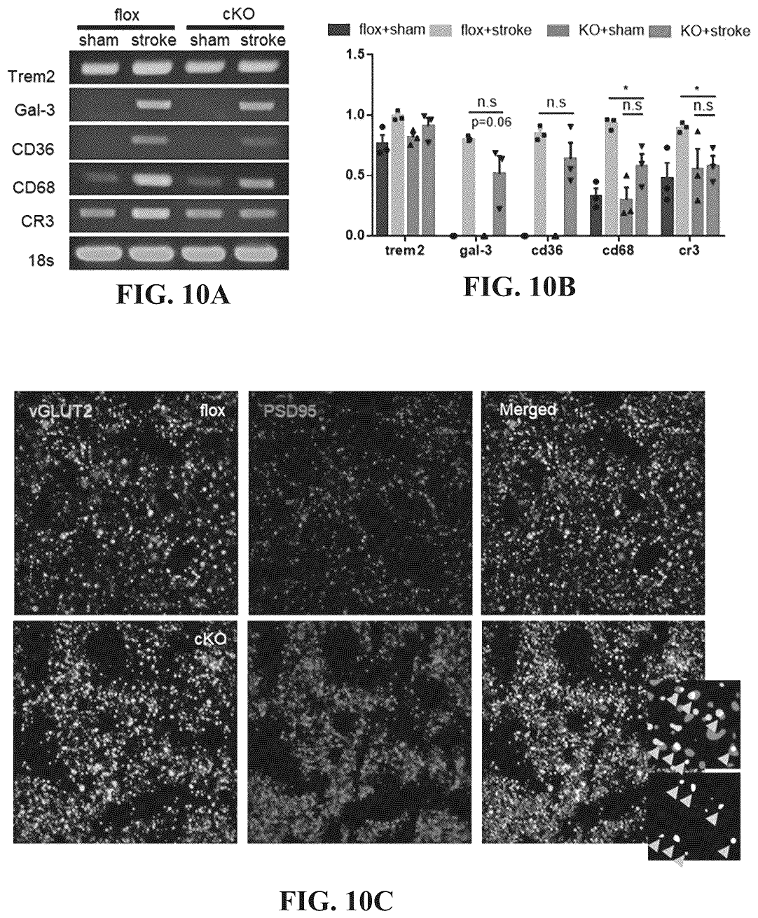

In animals deficient in the expression of arginase-1, it was confirmed that the expression of the corresponding genes, which increased after stroke, was reduced (FIGS. 10A and 10B). In addition, the synapse structure was confirmed through tissue immunostaining to determine whether these changes were actually involved in synapse formation after stroke. As a result, it was confirmed that the number of synapses represented by vglut2 and PSD95 was reduced in arginase-1 deficient animals (FIG. 10C). This confirmed that the decrease in synapses around the lesion after stroke was due to a decrease in microglia phagocytosis activity in arginase-1 deficient animals.

1. A pharmaceutical composition for preventing or treating ischemic cerebrovascular disease, comprising an arginase-1 inhibitor as an active ingredient.

2. The pharmaceutical composition of claim 1, wherein the expression of arginase-1 is expressed within infiltrating macrophages.

3. The pharmaceutical composition of claim 1, wherein the expression of arginase-1 is suppressed by deleting a gene encoding arginase-1 protein or inhibiting the function of arginase-1 protein.

4. The pharmaceutical composition of claim 3, wherein a substance that defects in the expression of the arginase-1 gene or inhibits the function of the arginase-1 protein is selected from the group consisting of DNA, RNA, polypeptides, proteins, ligands, enzymes, antibodies, antigens, natural compounds, synthetic compounds and bioactive molecules

5. The pharmaceutical composition of claim 1, wherein the arginase-1 inhibitor reduces the expression rate of inflammatory cytokines.

6. The pharmaceutical composition of claim 5, wherein the expression rate of the inflammatory cytokines is measured in microglia.

7. A method for screening a substance for preventing or treating ischemic cerebrovascular disease, comprising the steps of:
(a) treating stroke-inducing mice with a test substance to induce the deletion of a gene encoding arginase-1 protein or the inhibition of function of arginase-1 protein;
(b) measuring the inflammatory cytokine expression rate in stroke-induced mice in which a gene in the arginase-1 protein is defective or the function of the arginase-1 protein is inhibited; and
(c) selecting a candidate substance that reduces the expression rate of inflammatory cytokines in stroke-induced mice that are treated with the test substance compared to stroke-induced mice that are not treated with the test substance.

8. The method of claim 7, wherein the test substance is selected from the group consisting of DNA, RNA, polypeptides, proteins, ligands, enzymes, antibodies, antigens, natural compounds, synthetic compounds and bioactive molecules.

9. The method of claim 7, wherein the inflammatory cytokines can be measured in microglia.
